(19) United States
(12) Patent Application Publication
(10) Pub. No.: US 2018/0161815 A1
(43) Pub. Date: Jun. 14, 2018

(54) VIBRATOR ASSEMBLY COMPRISING ROTATION CONNECTION UNIT

(71) Applicant: Ligasonix, Inc., New York, NY (US)

(72) Inventor: Eungkook Kim, Seoul (KR)

(21) Appl. No.: 15/539,099

(22) PCT Filed: Dec. 22, 2015
(86) PCT No.: PCT/IB15/02603

§ 371 Date: Sep. 14, 2017

(30) Foreign Application Priority Data
Dec. 22, 2014 (KR) 10-2014-0186232

(51) Int. Cl. B06B 1/06; A61B 17/32; B06B 1/02

(52) U.S. Cl. CPC B06B 1/0611; A61B 17/320068; A61B 2017/00477; B06B 1/0207; A61B 17/320092

(57) ABSTRACT

The transducer assembly having a rotational connection unit according to the present invention comprises: a piezoelectric element which applies oscillation to a rotating body, and is provided to rotate together with the rotating body; a first conductor that is provided to have a greater inner diameter than the diameters of the piezoelectric element and an inner diameter portion that contacts one face of the piezoelectric element, comprises an outer diameter portion which encircles the circumference of the inner diameter portion, and which is provided to rotate together with the rotating body, and; a pair of second conductors that contact one face of the outer diameter portion of the first conductor and the other face thereof, respectively, have pass-through holes having a diameter greater than that of the piezoelectric element, and are connected to wiring supplying electrical power to deliver electrical power to the piezoelectric element through the first conductor.

### TECHNICAL FIELD

The present invention relates to a transducer assembly having a rotational connection unit. More specifically, the present invention relates to a transducer assembly that has a rotational connection unit which can freely rotate while supplying electrical power to a piezoelectric element, thereby preventing twisting of wiring.

### BACKGROUND ART

Special devices such as slip ring or drum/cylinder-type rotational electrical connectors to prevent twisting of wiring due to rotation while supplying electrical power to a rotating body are being widely used. The slip ring is provided in a form wherein separate electrode rings on discs facing each other rotate and maintain an electrical connection, or in a form where an outer ring and an inner ring that rotates within the outer ring are provided, with the outer ring and inner ring constantly in contact to maintain an electrical connection.

However, as such slip rings have a dual structure, their volume increases greatly, and the complexity of the structure thereof causes the problem of elevated fabrication costs.

Accordingly, slip rings are difficult to use in small devices, and even if they can be used, they greatly increase the unit price of products, making them difficult to use.

Another type of rotating electrical connection unit comes in a form involving gathering alternating same-polarity electrodes and connecting them to a sturdy and thick ring, and maintaining an electrical connection by pressing the respective poles using two electrode springs.

However, with such rotating electrical connection units, a small contact surface area means that a weak pressing force causes the electrical connection to be unstable. Further, if an emphasis is placed on maintaining the electrical connection to address this problem, resistance increases when rotating, preventing smooth rotation.

Accordingly, a method to address these problems is called for.

### DETAILED DESCRIPTION OF THE INVENTION

### Technical Problem

The purpose of the present invention, which has been conceived to solve the problems of prior art described in the above, is to provide a transducer assembly having a rotational connection unit which can freely rotate with low resistance while supplying electrical power to a piezoelectric element to prevent twisting of wiring, and which can be used in small devices.

The purpose of the present invention shall not be limited to the purposes mentioned in the foregoing, and other purposes not mentioned shall be clearly understood by a person having ordinary skill in the art from the following.

### Technical Solution

The transducer assembly having a rotational connection unit of the present invention, to achieve the above-stated purposes, comprises: a piezoelectric element which applies oscillation to a rotating body, and is provided to rotate together with the rotating body; a first conductor that is provided to have a greater inner diameter than the diameters of the piezoelectric element and an inner diameter portion that contacts one face of the piezoelectric element, comprises an outer diameter portion which encircles the circumference of the inner diameter portion and which is provided to rotate together with the rotating body, and; a pair of second conductors that contact one face of the outer diameter portion of the first conductor and the other face thereof, respectively, have pass-through holes having a diameter greater than that of the piezoelectric element, and are connected to wiring supplying electrical power to deliver electrical power to the piezoelectric element through the first conductor.

Further, the rotational connection unit includes a plurality of piezoelectric elements separate from each other, where a pair of adjacent piezoelectric elements may contact one face and the other face of the first conductor, respectively.

Also, the rotational connection unit may be provided to have an inner diameter greater than the outer diameter of the piezoelectric element, with an accommodating hole that accommodates the piezoelectric element, and may further comprise insulating members which prevent a pair of second conductors disposed on either side of the piezoelectric element from contacting one another.

Further, the first conductor may have slit holes forming at least one connection between the inner diameter portion and the outer diameter portion, where the inner diameter portion may move forward and backward within a certain range corresponding to the oscillations of the piezoelectric element.

Also, the second conductors may include projections which protrude from the circumference of the second conductors and which are connected to the wiring.

Also, the first conductor and second conductor are disposed on either side of the piezoelectric element, with a pair of second conductors disposed on either side of the piezoelectric element provided so that their respective projections are not aligned.

Further, a pass-through hole through which the rotating body passes through may be formed at the center of the piezoelectric element and the first conductor.

Also, the rotational connection unit may further comprise finishing members provided on one end and the other end of the rotational connection unit.

### Benefits of the Invention

The transducer assembly having a rotational connection unit of the present invention conceived to solve the problems described in the above has the following benefits.

The first advantage is that twisting of wiring is prevented while supplying electrical power to the piezoelectric element, allowing for free rotation.

The second advantage is that due to its structural characteristics, it can be used in small devices as well.

The third advantage is that due to the contact of the first conductor and the second conductor over a large area, the occurrence of failures can be minimized.

The fourth advantage is that the oscillations of the piezoelectric element are not reduced, and therefore maximum performance of the device can be had.

The benefits of the present invention shall not be limited to those benefits mentioned in the foregoing, and a person having ordinary skill in the art shall clearly comprehend other benefits not mentioned from the statements of the claims.

### BRIEF DESCRIPTION OF DRAWINGS

FIG. 1 is a skew drawing depicting the appearance of a medical apparatus with the transducer assembly according to one embodiment of the present invention;

FIG. 2 is a skew drawing depicting the transducer assembly according to one embodiment of the present invention provided within the medical apparatus;

FIG. 3 is a cross-sectional view illustrating the structure of the transducer assembly according to one embodiment of the present invention;

FIG. 4 is an exploded skew drawing showing the connection relationships among the respective components of the rotational connection unit of the transducer assembly according to one embodiment of the present invention;

FIG. 5 is a front view showing the components of the rotational connection unit of the transducer assembly according to one embodiment of the present invention in detail;

FIG. 6 is a side view showing the appearance of the first conductor of the transducer assembly according to one embodiment of the present invention;

FIG. 7 is a side view showing the assembled appearance of the rotational connection unit of the transducer assembly according to one embodiment of the present invention, and;

FIG. 8 is a cross-sectional view showing the structure of the rotational connection unit of the transducer assembly according to one embodiment of the present invention in detail.

### BEST MODE FOR CARRYING OUT THE INVENTION

In the following, preferable embodiments of the present invention by which the purposes of the present invention may be specifically realized shall be explained with reference to the attached drawings. In explaining these embodiments, the same names and same symbols shall be used for the same components, and additional explanation of such components shall be omitted.

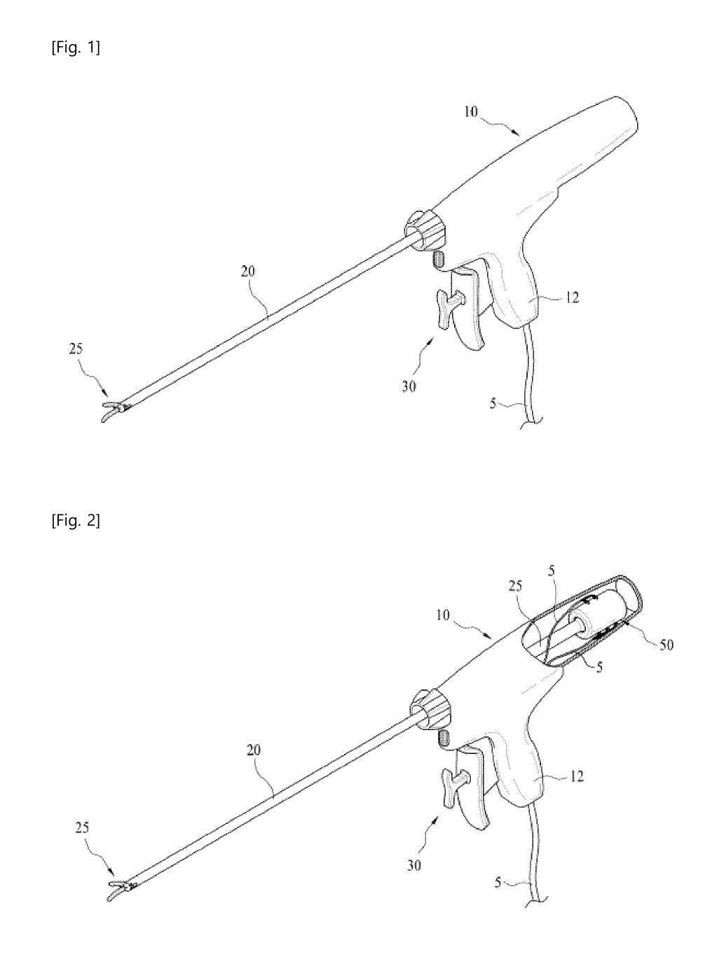

FIG. 1 is a skew drawing depicting the appearance of a medical apparatus with the transducer assembly according to one embodiment of the present invention, and FIG. 2 is a skew drawing depicting the transducer assembly according to one embodiment of the present invention provided within the medical apparatus.

As illustrated in FIG. 1 and FIG. 2, the transducer assembly according to one embodiment of the present invention is used in a medical apparatus. Provided, that this is shown as an embodiment only, and the transducer assembly may be used in a variety of rotating structures not restricted hereto.

The medical apparatus is an ultrasonic wave surgery apparatus, which is able to carry out procedures such as cutting and cauterizing of tissues such as blood vessels. Examining the apparatus, it comprises a body (10), a handle (12) provided on the body (10), a start button (30), a rod (20), and an effector portion (25).

Also, on the inside of the body (10) is provided the rotational connection unit (50) according to one embodiment of the present invention, where the rotational connection unit (50) is connected to the rod (20) to deliver ultrasonic oscillations to the effector portion (25), and receives electrical power for generation of ultrasonic oscillations from wires (5).

Here, the rod (20) is provided to be able to axially rotate with respect to a central point so as to facilitate the surgical process, and accordingly the rotational connection unit (50) has a structure that is able to maintain and electrical connection while preventing twisting of the wires (5). Such will be explained in detail in the following.

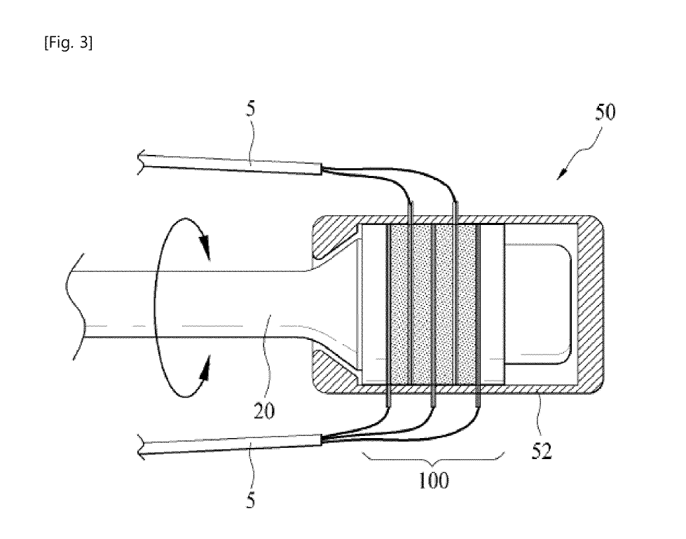

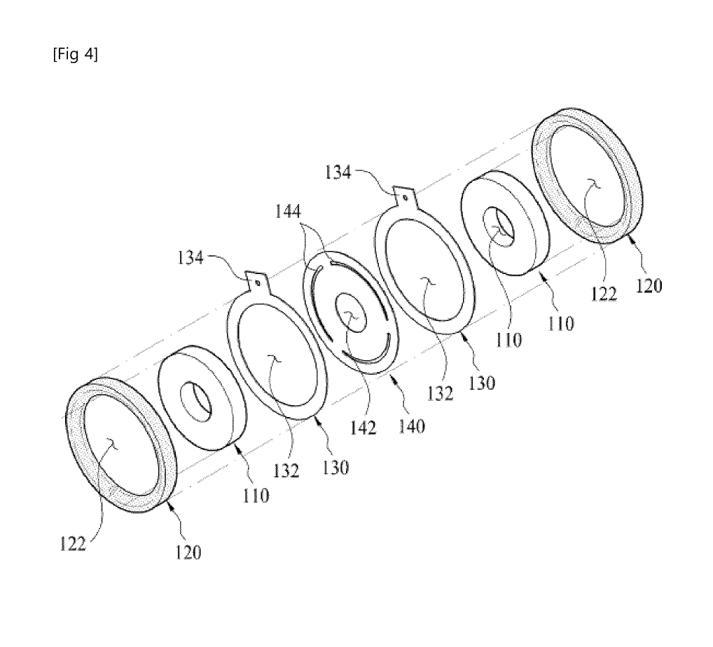

FIG. 3 is a cross-sectional view illustrating the structure of the transducer assembly (50) according to one embodiment of the present invention.

As illustrated in FIG. 3, in the case of the present embodiment, the transducer assembly (50) has a rotational connection unit (100) within a housing (52), and the rod (20) is provided penetrating the rotational connection unit (100).

Also, as the wiring (5) is also connected to the rotational connection unit (100), some components of the rotational connection unit (100) are provided to rotate together with the rod (20), while other components are connected to the wiring (5) and do not rotate but are fixed.

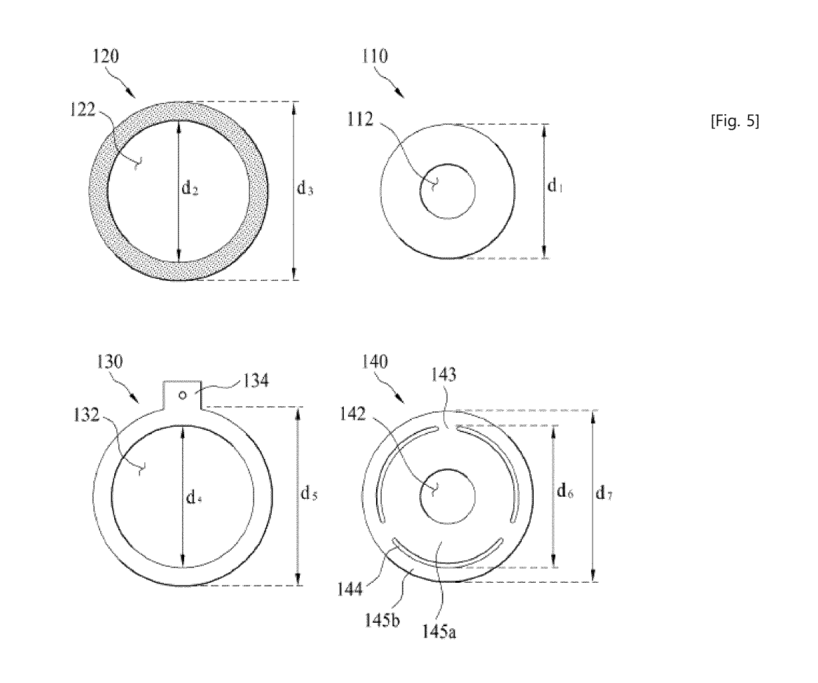

FIG. 4 is an exploded skew drawing showing the connection relationships among the respective components of the rotational connection unit of the transducer assembly according to one embodiment of the present invention, and FIG. 5 is a front view showing the components of the rotational connection unit of the transducer assembly according to one embodiment of the present invention in detail.

As illustrated in FIG. 4 and FIG. 5, in the present embodiment, the rotational connection unit comprises a piezoelectric element (110), a first conductor (140), a second conductor (130), and an insulating members (120).

The piezoelectric element (110) applies oscillations to the rotating body, that is, the rod described above in the case of the present embodiment (20, see FIG. 3), and is provided to rotate together with the rotating body. Various types of piezoelectric element may be used as the piezoelectric element, and in the present embodiment, [the piezoelectric element] generates oscillations when voltage is applied.

The first conductor (140) comprises an inner diameter portion (145a) that contacts one face of the piezoelectric element (110), and an outer diameter portion (145b) that encircles the circumference of the inner diameter portion (145a). Here, as the outer diameter portion (145b) is provided to have an inner diameter (d₆) greater than the outer diameter (d₁) of the piezoelectric element (110), the piezoelectric element (110) can be in contact with the inner diameter portion (145a) only without contacting the outer diameter portion (145b).

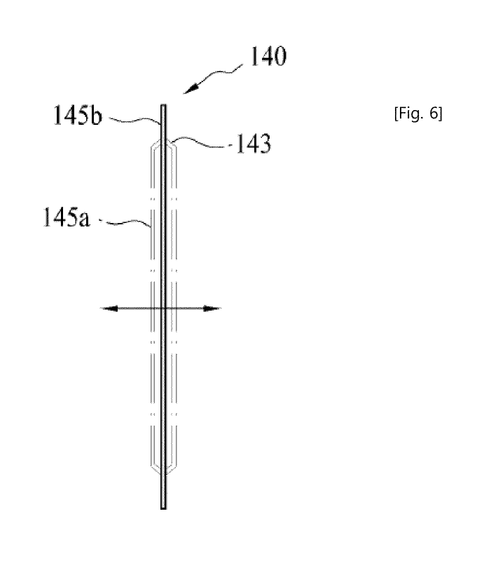

Also, in the case of the present embodiment, the first conductor (140) has slit holes (144) forming at least one connection (143) between the inner diameter portion (145a) and the outer diameter portion (145b). Accordingly, the inner diameter portion (145a) and the outer diameter portion (145b) are separated from each other, excluding the connections (143), and the inner diameter portion (145a), as shown in FIG. 6, may be provided to move forward and backward within a certain range corresponding to the oscillations of the piezoelectric element (110). Accordingly, the first conductor (140) naturally corresponds to the oscillations of the piezoelectric element (110), becoming structurally stable.

Also, as such first conductor (140) is also in contact with the piezoelectric element (110), it can rotate with the rotations of the rotating body.

A pair of second conductors (130) is provided, contacting one face and the other face of the outer diameter portion (145b) of the first conductor (140), respectively. That is, a pair of second conductors (130) are in contact with both sides of the first conductor (140).

Here, on the second conductor (130), a pass-through hole (132) with a diameter (d₄) greater than the outer diameter (d₁) of the piezoelectric element (110) is provided. Accordingly, the second conductor does not contact the piezoelectric element (110), and contacts only the outer diameter portion (145b) of the first conductor (140).

Also, in the present embodiment, the second conductor (130 includes projections (134) which project from the circumference thereof and connect to the wiring described above, and are able to receive electrical power from the wiring to be delivered to the piezoelectric element (110) via the first conductor (140). Also, as the second conductor (130) is connected to the wiring, it does not rotate but remains fixed.

Due to such structure, when the rotating body rotates, the piezoelectric element (110) and the first conductor (140) rotate together, and here, slippage occurs between the areas of the first conductor (140) and the second conductor (130) that are in contact with each other, causing no resistance against rotation. Here, as the first conductor (140) and the second conductor (130) remain in contact through a broad, disc-shaped contact area greater than the outer diameter of the piezoelectric element (110), the electrical connection can be continuously maintained.

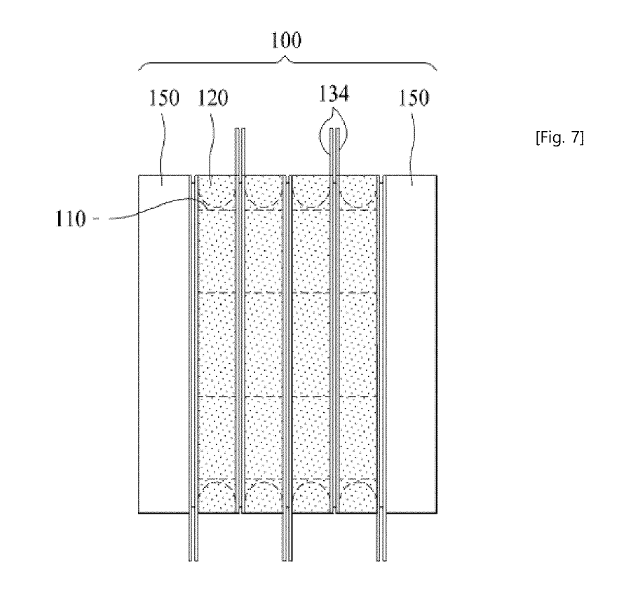

Meanwhile, in the case of the present embodiment, as shown in FIG. 7, a plurality of piezoelectric elements (110) separated from each other are provided. Accordingly, a common structure of a first conductor (140) and a pair of second conductors (130) is provided between the respective piezoelectric elements (110). This shall be explained later.

Meanwhile, in such case, to keep the second conductors (130) provided between the respective piezoelectric elements (110) from moving about freely and coming into contact with each other, insulating members (120) may be further provided.

Referring again to FIG. 5, the insulating member (120) is provided to have an inner diameter (d₂) greater than the outer diameter (d₁) of the piezoelectric element (110), and has an accommodating hole (122) to accommodate the piezoelectric element (110) so that the piezoelectric element (110) does not cause resistance when rotating. Accordingly, the insulating member (120) can keep the pair of second conductors (130) provided on either side of the piezoelectric element (110) from coming into contact with one another.

Also, in the case of the present embodiment, the outer diameter (d₃) of the insulating member (120) and the outer diameter of the second conductor (130) correspond to the inner diameter of the housing described in the foregoing (52, see FIG. 3), and are inserted into the housing (52), with the outer diameter (d₇) of the first conductor (140) being smaller that these to allow for smooth rotation.

In addition, in the present embodiment, a pass-through hole (112, 142) through which the rotating body passes through is provided at the centers of the piezoelectric element (110) and the first conductor (140).

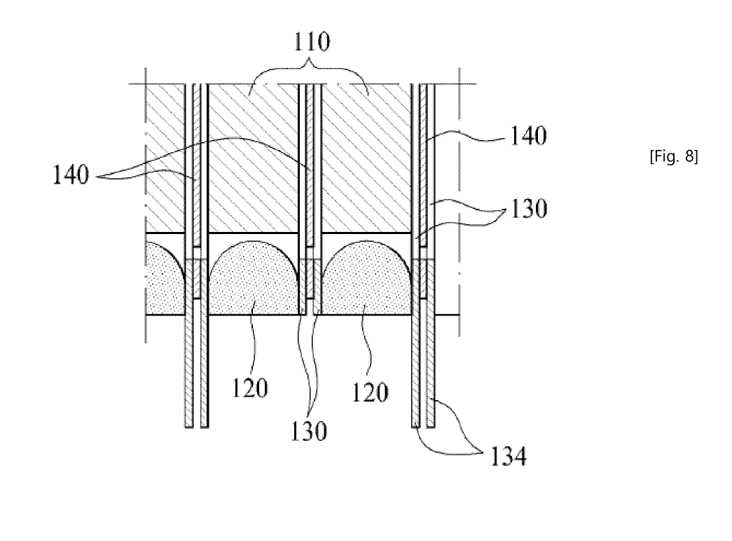

Meanwhile, in the case of the present embodiment, as shown in FIG. 7 and FIG. 8, the rotational connection unit (100) may further comprise a pair of finishing members (150) provided on one end and the other end of the rotational connection unit (100). The finishing members (150) perform the roles of protecting and holding in place the piezoelectric element (110), the first conductor (140), the second conductor (130), and insulating members (120) provided between a pair of finishing members (150).

Also, as explained in the foregoing, in the present embodiment, between neighboring piezoelectric elements (110) and between the piezoelectric element (110) and the finishing member (150) are provided one first conductor (140) and a pair of second conductors (130) provided on either side of the first conductor (140), respectively.

As a total of 4 piezoelectric elements (110) are provided in the present embodiment, a total of 5 groups of one first conductor (140) and a pair of second conductors (130) are provided.

Also, as for the projections (134) provided on the second conductors (130), the direction in which they project alternate between groups. Accordingly, the projections (134) projecting in one direction and the projections projecting in another direction (134) are electrically connected by different wiring (5, see FIG. 3), and form different poles, respectively.

Provided, that this is only one embodiment, and of course the outermost group among the 5 groups may be omitted.

As described in the foregoing, the transducer assembly according to the present invention is able to rotate freely while supplying electrical power to a piezoelectric element (110), preventing twisting of wiring.

Although the specific exemplary embodiments have been described and illustrated as above, the present invention is not limited to the exemplary embodiments described herein, and it would be apparent to those skilled in the art that various changes and modifications might be made to these exemplary embodiments without departing from the spirit and the scope of the invention. Accordingly, the changed example and modified examples should not be individually appreciated from the technical spirit or the viewpoint of the present invention and it should be appreciated that modified exemplary embodiments will be included in the appended claims of the present invention.

### EXPLANATION OF SYMBOLS

50: Transducer assembly

52: Housing

100: Rotational connection unit

110: Piezoelectric element

112: Pass-through hole

120: Insulating member

122: Accommodating hole

130: Second conductor

132: Pass-through hole

134: Projection

140: First conductor

142: Pass-through hole

143: Connection

144: Slit hole

145a: Inner diameter portion

145b: Outer diameter portion

150: Finishing member

What is claimed is:

1. A transducer assembly having a rotational connection unit comprising: a piezoelectric element which applies oscillation to a rotating body, and is provided to rotate together with the rotating body;
a first conductor that is provided to have a greater inner diameter than the diameters of the piezoelectric element and an inner diameter portion that contacts one face of the piezoelectric element, comprises an outer diameter portion which encircles the circumference of the inner diameter portion, and which is provided to rotate together with the rotating body, and;
a pair of second conductors that contact one face of the outer diameter portion of the first conductor and the other face thereof, respectively, have pass-through holes having a diameter greater than that of the piezoelectric element, and are connected to wiring supplying electrical power to deliver electrical power to the piezoelectric element through the first conductor.

2. The transducer assembly according to claim 1, wherein the rotational connection unit includes a plurality of piezoelectric elements separate from each other, where a pair of adjacent piezoelectric elements contact one face and the other face of the first conductor, respectively.

3. The transducer assembly according to claim 2, wherein the rotational connection unit is provided to have an inner diameter greater than the outer diameter of the piezoelectric element, with an accommodating hole that accommodates the piezoelectric element, and further comprises insulating members which prevent a pair of second conductors disposed on either side of the piezoelectric element from contacting one another.

4. The transducer assembly according to claim 1, wherein the first conductor has slit holes forming at least one connection between the inner diameter portion and the outer diameter portion, where the inner diameter portion can move forward and backward within a certain range corresponding to the oscillations of the piezoelectric element.

5. The transducer assembly according to claim 1, wherein the second conductors include projections which protrude from the circumference of the second conductors and which are connected to the wiring.

6. The transducer assembly according to claim 5, wherein the first conductor and second conductor are disposed on either side of the piezoelectric element, with a pair of second conductors disposed on either side of the piezoelectric element provided so that their respective projections are not aligned.

7. The transducer assembly according to claim 1, wherein a pass-through hole through which the rotating body passes through is formed at the center of the piezoelectric element and the first conductor.

8. The transducer assembly according to claim 1, wherein the rotational connection unit further comprises finishing members provided on one end and the other end of the rotational connection unit.
